(19) United States
(12) Patent Application Publication
(10) Pub. No.: US 2023/0148922 A1
(43) Pub. Date: May 18, 2023

(54) SYSTEMS AND METHODS FOR SCREENING SUBJECTS FOR NEUROPATHOLOGY ASSOCIATED WITH COVID-19 UTILIZING A MOBILE DEVICE

(71) Applicant: Great Plain Technologies LLC, Pittston, PA (US)

(72) Inventors: Clifton R. LACY, Highland Park, NJ (US); Warren S. Gifford, Monroe Township, NJ (US); David L. Turock, Fort Lauderdale, FL (US)

(21) Appl. No.: 17/527,356

(22) Filed: Nov. 16, 2021

(51) Int. Cl. A61B 5/16; A61B 5/00

(52) U.S. Cl. CPC A61B 5/163; A61B 5/6898; A61B 5/742; A61B 5/746

(57) ABSTRACT

Systems and methods for screening a subject for neuropathology associated with COVID-19. The method can include providing neurophysiological stimuli to the subject, measuring response by the subject to the neurophysiological stimuli, comparing the measured responses to a reference, determining whether the subject demonstrates abnormal or changed responses to the neurophysiological stimuli according to whether the measured responses differ from the individual's baseline or from the reference by one or more thresholds, and providing an alert according to whether the subject has abnormal or changed responses to the neurophysiological stimuli, wherein the alert comprises an intervention associated with COVID-19.

### BACKGROUND

### Eye Movement

Eye movement characteristics can be a useful tool to help differentiate normal function from that of neuropathology including mild cognitive impairment found in COVID-19 and other disorders, such as dementia, Alzheimer's disease, and traumatic brain injury. Additional uses include assessments of individuals with autism, depression, schizophrenia, vertigo, nystagmus, and epilepsy. Video-oculo-graphic recordings of eye movements are an objective means for quantitatively assessing ocular motor performance. These findings can be sensitive biomarkers, both as a current “snapshot” of status and as a longer-term history of performance.

### Eye Tracking

Eye tracking techniques objectively measure, in space and time, both the position of the eyes and their movement. Testing involves assessment of overall eye tracking, attention to target, dwell time on target, and other measured gaze variables including fixations, pursuits, saccades, gaze shifts, visual searching, and social cognition. These can be performed with or without specialized head-mounted hardware and can be accomplished without the need to have fixation of the head in place. Eye movement can be recorded as individual images or in video format to which artificial intelligence/machine learning methods can be applied for big data analysis, data mining, and more refined classification of normal and abnormal responses. In addition to yielding information regarding oculomotor muscle function, eye tracking allows characterization of target scanning errors including distraction/disinhibition errors, staring/perseverative errors, and order/sequencing errors which allow more refined evaluation of cognitive dysfunction in neurological disorders.

The measured quantitative parameters can serve as noninvasive markers for change in cognition and detection of cognitive impairment, such as seen in individuals with Long COVID.

### Trail-Making

Trail-making tests (TMTs) are a well-established methodology for neuropsychological assessment of cognitive processes including visual attention, visual search and motor scanning, sequencing and task-switching, psychomotor processing speed, and ability to execute a plan of action, as well as higher level cognitive skills such as mental flexibility.

TMTs are timed measurements instructing a subject to connect numbers and/or letters in numerical and/or alphabetical order, in forward, backward, or alternating fashion by eye motion, finger pointing, tapping on the screen, and other methods.

TMTs reflect cognitive abilities of speed and fluid intelligence and can be employed as a current “snapshot” to identify cognitive impairment such as seen in Long COVID and, when compared with baseline determinations, can detect and track deterioration or improvement over time by degree of impairment. In addition, for certain conditions like amyotrophic lateral sclerosis, motor neurons serving ocular function are largely preserved, allowing testing by eye motion TMT that would be otherwise impossible to do with skeletal muscle function.

### Eye-Hand Coordination

Eye-hand coordination (EHC) is the interconnected relationship between visual and manual motor systems. Visually guided object interaction requires visual detection and motor coordination of the hand to produce intentional, controlled, timed, and accurate movements. Measurement of spatial and temporal responses can be of use to detect impairment and monitor progression or improvement of disease.

Eye-hand coordination testing can be used as a tool to assess neurological disorders and conditions. For example, a decline in visual-manual motor functions has been demonstrated in early stages of neurodegenerative disorders such as Alzheimer's disease and Parkinson's disease. Impairment in eye-hand coordination has been reported in adolescents with Long COVID.

### Smart Device Technology

Smart mobile device technology has been employed to assess cognitive impairment and neurological disorders. These methods facilitate single, repeated, and continuous cognitive assessment for characterization of individual status and change, as well as allowing population and norm-based comparisons. The methods allow detection of gradual changes which may otherwise be difficult to identify, as well as acute changes which may indicate a need for urgent intervention. Although most medical investigations for characterization of cognitive and other neurological abnormalities involve invasive, time-consuming, expensive, and sometimes difficult-to-access technologies, smart phone-based assessments can be performed in the comfort of one's own home or at mass testing kiosks. Smartphone-based mobile technology makes assessment easier for those who are infirmed, have physical disabilities, and/or are older. Ease of assessment is becoming increasingly important as the number of people diagnosed with cognitive impairment is growing rapidly as the population ages and as more individuals sustain concussive head injuries. It has been shown that the majority of older adults own or have ready access to a smart phone.

### Cognitive Function

Cognitive function includes learning and memory; language; and visuospatial, executive, and psychomotor function. Cognitive decline is frequently undiagnosed until daily functioning is disrupted. Early detection of cognitive decline can guide intervention to promote retention and, in some cases, improvement in cognitive functioning. Tests facilitating early detection of cognitive decline would be of great value nationally and internationally.

### Delirium Versus Dementia

The neurological conditions of delirium and dementia can be differentiated by a number of characteristics including onset timing, pace of deterioration, duration, course and timing of resolution or permanency, associated attention deficit, level of consciousness, orientation to time/place/person, disturbances of language and speech, and preservation or loss of memory, among others.

### Additional Methods of Cognitive Function Testing

Smart Device Technology can include testing for immediate recognition, semantic memory, categorization, subtraction, repeating backward, clock drawing, cube copy, cube rotation, pyramid rotation, trail-making, delayed recognition tests, symbol matching tasks, memory tasks, and object matching tasks to assess a variety of aspects of cognitive function, including concentration, memory, and visuospatial function.

Decline in cognitive function can be assessed through changes in physical movement. GPS data focusing on geographic area and perimeter of activities of daily life can be used as indicators of both physical and cognitive function. The area and perimeter coverage within and outside the home can be used to distinguish between healthy people and those with cognitive impairments like dementia. Accelerometer-derived gait velocity can yield information about disorders of affective status such as depression. Assessment of fine motor skills from tapping on the screen; movement of right, left, or both hands in time with audio signals; and measures of tap response time, rhythm, and contact duration, and inter-hand divergence can be used to assess for dementia and mild cognitive impairment.

Analysis of speech and vocal characteristics employed for cognitive assessment include vocal cognitive tasks such as sentence repetition, denomination, picture description, verbal fluency phonemic, verbal fluency sematic, counting backward, and positive/negative/episodic storytelling.

Sound can be used separately or in conjunction with other neurophysiological stimuli to elicit a response, distract, instruct, and inform.

### Long COVID

Long COVID is defined as symptoms associated with COVID-19 that persist weeks or months after the acute illness. Studies suggest that as many as 30% of individuals with suspected or confirmed COVID-19 have persistent symptoms, including those who were asymptomatic or mildly symptomatic with the acute infection. In fact, some studies have found as many as 91% of patients, whether or not manifesting neurological problems when hospitalized, had persistent neurological issues 6 months after discharge. Studies have reported “brain fog” as a major symptom, with about 50% demonstrating impaired cognition, 47% unable to return to work, and many having abnormal levels of anxiety, sleep disturbance, generalized fatigue, and depression at 6 months after acute disease.

The term “COVID-19 brain fog” refers to cognitive impairments characterized by an inability to concentrate, sustain attention, remember, or think or reason clearly.

Postulated causes of “COVID-19 brain fog” include brain cell infection (from SARS-CoV-2) or inflammation (due to the virus or an autoimmune disorder); brain ischemia (lack of blood flow and oxygen) due to brain blood vessel-associated edema (swelling), occlusion, and/or hemorrhage; hypoxemia (low blood oxygen levels) from lung damage; or secondary damage to the heart, kidneys, or liver.

### SUMMARY

There are provided systems and methods for screening subjects for neuropathological conditions, particularly by tracking subjects' responses to neurophysiological stimuli, including visual and audio stimuli.

Described herein are various embodiments of systems and methodologies to detect and characterize subtle and non-subtle cognitive and other neurophysiological disturbances in an individual. The systems described herein utilize single and multiple (over-time) determinations of neurophysiological assessment with comparison to the individual's baseline determinations, as well as to population-derived values, to detect and characterize incipient or established abnormalities of response. These findings then trigger notification of the individual to seek professional medical attention for evaluation and further workup and treatment as appropriate. Accordingly, the systems and methodologies can detect neurological findings associated with Long COVID. In addition to Long COVID, the systems and methodologies described herein are further able to detect other neuropathologies, which can assist in referral for definitive medical diagnosis and treatment. Because there are no standard, easily administered tests for frequent screening that are currently available, the systems further provide a platform for testing alternative testing protocols and collecting the data needed for research and for identification and deployment of best practices.

As noted above, eye tracking, trail-making, eye-hand coordination, and other testing techniques that utilize smart device technology allow for the identification of cognitive impairment and other neuropathological conditions, such as seen in Long COVID. When such tests are performed once, they can be used to detect an acute instance of a neuropathological condition. When performed on multiple occasions, test results can be compared with baseline determinations to detect and track deterioration or improvement of a neuropathological condition over time. Further, the systems can be used to screen subjects for neuropathological conditions, regardless of whether the individuals exhibit conventional or acute symptoms of the conditions. Further, if the testing provided by the systems indicates that an individual is developing new symptoms of cognitive impairment or there is a rapid progression of symptoms of cognitive impairment, the system can refer the individual for formal medical evaluation for determination of COVID-19 or other neurological disorders. Because the systems are able to iteratively perform the testing techniques on an individual over a period of time, the systems can identify newly developed symptoms or the rapid onset of symptoms associated with neuropathologies, even if they do not exhibit conventional or obvious symptoms associated with “brain fog.”

A system that could screen for findings of COVID-19 in an accurate, reliable and quantitative manner based on individuals' responses (or lack thereof) to neurophysiological stimuli, including visual and audio stimuli, would benefit the individuals themselves and provide a significant public health benefit. Additionally, a variety of other conditions (e.g., multiple sclerosis, Alzheimer's disease and other forms of dementia) are associated with neuropathology. Therefore, such a system could also be used to screen for findings associated with these types of conditions.

In some embodiments, there is provided a computer-implemented method for screening a subject for a response to neurophysiological stimuli as an indication for neuropathology associated with a condition, the method comprising: presenting, by a display screen of a mobile device, the neurophysiological stimuli to the subject; measuring, by a detector of the mobile device, a neurophysiological response by the subject to the neurophysiological stimuli; determining, by the processor, whether the measured neurophysiological response differs from a reference response by a threshold, whereby such a difference comprises an abnormal response; and providing, by the processor, an alert for the abnormal response, wherein the alert comprises an intervention associated with a condition.

In some embodiments, a system for screening a subject for a neurophysiological response to neurophysiological stimuli as an indication for neuropathology associated with a condition, the system comprising: a library storing the neurophysiological stimuli; and a mobile device comprising: a display screen, a detector, a processor, and a memory coupled to the processor, the memory storing instructions that, when executed by the processor, cause the processor to: present, via the display screen, the neurophysiological stimuli to the subject, measure, via the detector, the neurophysiological response by the subject to the neurophysiological stimuli, determine whether the measured neurophysiological response differs from a reference response by a threshold, whereby such a difference comprises an abnormal response, and provide an alert for the abnormal response, wherein the alert comprises an intervention associated with the condition.

In some embodiments of the method and the system, the condition comprises COVID-19.

In some embodiments of the method and the system, the measured neurophysiological response comprises at least one of a change in speed, magnitude, or accuracy of the subject.

In some embodiments of the method and the system, the neurophysiological stimuli comprise at least one of an eye tracking test, a trail-making test, or an eye-hand coordination test.

In some embodiments of the method and the system, the neurophysiological response comprises at least one of an eye movement response or a hand motion response.

In some embodiments of the method and the system, the intervention comprises at least one of a recommendation to take a COVID-19 diagnostic test or a recommendation to seek medical evaluation.

In some embodiments of the method and the system, the reference response comprises one or more default values.

In some embodiments of the method and the system, the reference response comprises at least one of a characterized response or a baseline response to the neurophysiological stimuli for the subject.

In some embodiments of the method and the system, the reference response comprises at least one of a characterized response of a population of individuals to the neurophysiological stimuli.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 illustrates a diagram of a screening system, in accordance with an embodiment.

FIG. 2 illustrates a schematic diagram of a first embodiment of the screening system of FIG. 1.

FIG. 3 illustrates a schematic diagram of a second embodiment of the screening system of FIG. 1.

FIG. 4 illustrates a schematic diagram of a third embodiment of the screening system of FIG. 1.

FIG. 5 illustrates a diagram of another embodiment of the screening system.

FIG. 6 illustrates a flow diagram of a process for screening a subject for a condition via a neurophysiological stimulus, in accordance with an embodiment.

### DETAILED DESCRIPTION

As used herein, “COVID-19” means the infectious disease caused by the SARS-CoV-2 virus.

As used herein, “Long COVID” means symptoms associated with COVID-19 that persist weeks or months after the acute illness, such as brain fog.

As used herein, “brain fog” or “COVID-19 brain fog” means cognitive impairments resulting from COVID-19 that are characterized by an inability to concentrate, sustain attention, remember, or think or reason clearly.

As used herein, a “neuropathological condition” means a disease or physiological condition that exhibits or causes a neuropathological effect in a subject. Neuropathological conditions could include, for example, Long COVID or dementia.

As used herein, a “subject” refers to a human individual.

Generally described herein are various systems and processes for providing a subject with a visual stimulus and tracking the subject's response(s) to the visual stimulus in order to characterize and assess cognitive and other neurological disturbances in the subject over time. These systems and methods can be used for screening a subject for findings that indicate a reduction in the subject's neurological capabilities, which can in turn be indicative of particular symptoms associated with COVID-19 and/or Long COVID (e.g., brain fog) or other conditions, such as dementia. If the system identifies a change in a subject's neurological response or a deviation in the subject's neurological response relative to a baseline or a reference, the system could prompt the subject to seek medical evaluation, recommend further testing, recommend that the subject self-quarantine or isolate, or take a variety of other actions. Accordingly, the systems and processes described herein can be used to accurately, reliably, qualitatively, and quantitatively screen individuals for abnormal responses that may indicate a need for medical evaluation.

### Systems for Screening Subjects

Described herein are systems and techniques for providing neurophysiological stimuli to a subject and assessing the subject's response thereto in order to screen for one or more neuropathologies associated with COVID-19. In various embodiments, the neurophysiological stimuli could include audio stimuli or visual stimuli. Further, the neurophysiological stimuli could take the form of various testing techniques that are provided to the subject, including eye tracking, trail-making, or eye-hand coordination testing techniques. In one embodiment, a screening system 100 can include a visual stimulus source 112, a tactile response detector 102, and a visual response detector 104. The visual stimulus source 112 can include a standalone display screen or a display integrated into another device (e.g., a display of a smartphone). In one embodiment, the visual stimulus source 112 and the tactile response detector 102 can be integral to each other. For example, the visual stimulus source 112 and the tactile response detector 102 can be embodied as a touchscreen (e.g., a capacitive touchscreen). In one embodiment, the visual response detector 104 can include a camera or an image sensor. The visual response detector 104 can have sufficient resolution and other characteristics necessary to be able to detect the movements of a subject's eyes or portions thereof (e.g., the pupil) when positioned within a threshold distance to the subject.

The screening system 100 can be programmed or otherwise configured to provide a visual stimulus to a subject and track, monitor, or record the subject's response to the visual stimulus. Based on the subject's tracked response to the visual stimulus, the screening system 100 can be programmed or otherwise configured to make a determination as to whether the subject has findings compatible with a neuropathological condition, such as Long COVID or dementia. The visual stimulus provided to the subject could include a series of dots or patterns, icons, alphanumeric characters, or any other markers that can be visually identified and tracked by subjects. In various embodiments, the visual stimulus or portions thereof can move across the visual stimulus source 112, disappear and/or appear at various points on the visual stimulus source 112, change color, change shape, change visual perspective (e.g., rotate), and otherwise change in visually detectable manners. The screening system 100 can track a variety of different types of responses by a subject to the stimulus, including, for example, ocular responses, physical responses, or autonomic responses by the subject. In one embodiment, the screening system 100 can be configured to track eye movements by the subject in response to movements or changes by the visual stimulus. In another embodiment, the screening system 100 can be configured to track the ability of the subject to perform a trail-making test. As noted above, a trail-making test is a timed measurement of a subject's ability to connect numbers, letters, or other visual markers in a particular order (e.g., numerical or alphabetical order). A trail-making test can further task the subject with connecting the visual markers in a variety of different manners (e.g., in a forward, a backward, or an alternating fashion). The screening system 100 can track the subject's response(s) to the visual stimulus via the tactile response detector 102, the visual response detector 104, or a combination thereof depending upon the particular response being tracked thereby. For example, in embodiments where the response being tracked is the subject's eye movements, the screening system 100 can utilize the visual response detector 104 to track the characteristics of the subject's eyes. As another example, in embodiments where the response being tracked is the subject's ability to perform a trail-making test, the screening system 100 can utilize the tactile response detector 102 to track the subject's response to the trail-making test.

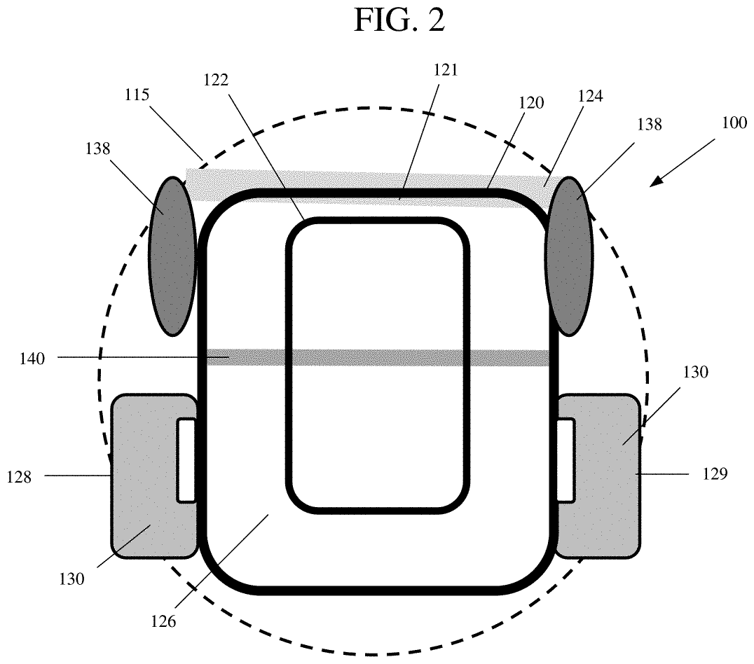

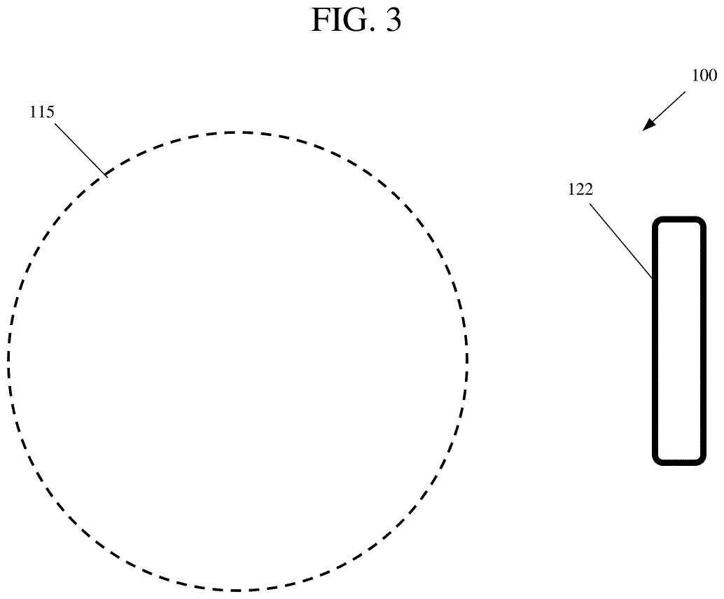

The visual response detector 104 could include standalone sensing devices or be incorporated into another device (e.g., a mobile device 122, as in the embodiments shown in FIGS. 2 and 3) or system. Further, in some embodiments, the visual response detector 104 could include one sensor or a set of sensors (i.e., a sensor assembly). The screening system 100 can be configured to execute various processes, such as those described below, to screen individuals based on their response or responses to stimuli provided by the screening system. In one embodiment, the screening system 100 can further include a processor 106 coupled to a memory 108 for storing data, including logic or instructions embodying processes to be executed by the processor.

The visual response detector 104 can be configured to capture images or video of a subject in sufficient detail such that the subject's eye movement response to the visual stimulus can be measured and, thus, quantified. In other words, the visual response detector 104 can be configured to capture images or video in a sufficiently high resolution and with sufficient clarity such that image processing algorithms can identify the subject's eyes (or portions thereof, such as pupils) and measure changes associated therewith. In various embodiments, the ocular response measured by the visual response detector 104 could include a change in the size (e.g., diameter or area) of the subject's pupil or pupils, timing information (e.g., hesitancy or delay in the pupil's movement), and other pupillary parameters. For example, the visual response detector 104 could be used to take a first measurement of a characteristic of the subject's eyes and take a second measurement of the characteristic after the subject has been provided the visual stimulus or after the initially provided visual stimulus has been changed by the screening system 100 (e.g., has moved, disappeared and reappeared at a different location on the visual stimulus source 112, changed in shape, or changed color). Accordingly, the subject's response to the visual stimulus could include the difference between the first and second measurements of the ocular characteristic.

In one embodiment, the screening system 100 could further include an auditory stimulus source 118. The auditory stimulus source 118 can be used to deliver instructions, produce audio signals for tapping cadence, be an element of the stimulus, or provide distractions as necessary for the testing.

The screening system 100 can be embodied as a variety of different objects, devices, or systems. In one embodiment, the screening system 100 could include a mobile device (e.g., a smartphone) and the processes executed thereby could include an app. In this embodiment, the screening system 100 could be beneficial by allowing individuals to self-screen for a particular condition or set of conditions using their own mobile device. In some embodiments, the visual response detector 104 could be embodied as an accessory or dongle that is connectable (either wirelessly or via a wired connection) or attachable to the mobile device. In other embodiments, the visual response detector 104 could include the onboard camera of the mobile device. Other embodiments could be suitable for screening individuals for entry to potentially crowded locations (e.g., schools, airports, or stadia). In one such embodiment, the screening system 100 could include a kiosk or station that includes the visual stimulus source 112 for providing the visual stimulus to subjects within the kiosk and the tactile response detector 102 and/or visual response detector 104. In this embodiment, the screening system 100 could be beneficial by allowing individuals to be screened for potential abnormalities (e.g., such as those associated with Long COVID or COVID-19 generally) prior to being permitted entry into a location. An abnormal response could be used as one of the tools to decide whether individuals should be permitted access to a venue, or require additional screening, thereby potentially avoiding significant adverse consequences (e.g., disease transmission events).

The screening system 100 can further include or be communicably connected to a database 110. The database 110 could include a local database 204 and/or a remote database 206, as described below. In one embodiment, the database 110 could be stored locally (i.e., in the memory 108). In another embodiment, the database 110 could be remote from the screening system 100. In this embodiment, the database 110 could be stored in a cloud computing storage system (e.g., Amazon Web Services), a remote server, and other such remote systems. The database 110 can be configured to store information including user parameters and settings, such as the user's previously calibrated responses. The user parameters could be embodied as a user profile, for example. The user parameters could include previously recorded values or measurements associated with the response measured by the screening system 100. The recorded parameters can be used to define a characterized or default response by the subject to the stimulus, which can in turn be used by the screening system 100 to determine when the subject's measured response deviates from this characterized or default response by the subject. Accordingly, the screening system 100 can determine when there has been a change in the patient's response to the stimulus, which could indicate that the patient has a condition that is screened by the screening system 100. The characterized or default response could be used to define various thresholds or ranges that could be used to determine whether the subject has passed or failed the screening. Accordingly, the screening system 100 can be configured to take measurements (e.g., via the visual response detector 104 and/or tactile response detector 102) associated with the subject's response to the stimulus, retrieve a user profile associated with the subject (e.g., from the database 110), and determine whether the subject has passed or failed the screening based on a comparison between the measurements of the response and the user profile parameters or a reference. For example, the screening system 100 can be configured to provide a trail-making test to the subject and measure the subject's response (i.e., the ability to properly track the moving visual stimulus) thereto. If there is a significant deviation from the subject's ability to perform the trail-making test relative to a reference or baseline (e.g., the stored, pre-characterized performance on the trail-making test associated with the subject or a universal characterized response), then the subject may be suffering from a neuropathological condition. Accordingly, the screening system 100 could prompt the subject regarding the need for medical evaluation (such as a physician checkup and/or testing including COVID-19 test) and/or suggest that the individual take corresponding appropriate precautions (e.g., self-quarantine or isolation). Conversely, if there is no significant deviation from the subject's ability to perform the trail-making test as compared to the reference or baseline, then the subject may not be suffering from such a neuropathological condition. Accordingly, the screening system 100 could take no action. In some embodiments, the screening system 100 can further record the subject's response(s) to the provided visual stimulus. The recorded responses could be used to further characterize the subject's baseline response characteristics, aggregated with records of other subjects (e.g., in the database 110) to further characterize universal or population-wide response characteristics, and so on.

The screening system 100 can further be configured to account for various secondary factors and be calibrated for each individual subject. For example, the screening system 100 may need to be calibrated to determine the baseline or expected response characteristics (e.g., eye movement hesitancy or degree of dilation in response to various visual stimuli) exhibited by the subject. Once the baseline or expected response characteristics are determined, subject measurements of those characteristics by the screening system 100 can be used to distinguish between potential neuropathological conditions and other abnormalities.

In one embodiment, the screening system 100 can be configured to determine the amount of light in the patient's environment (e.g., via the visual response detector 104) and, accordingly, account for the amount of light employed to determine the subject's response to the screening. The amount of ambient or environmental light can be an important factor because it can affect the ability of the subject's eyes to properly identify and distinguish between various visual stimuli, the contraction and dilation of the subject's pupils, and so on. In particular, a brightly lit ambient environment could decrease the pupil opening and, because the pupil is constricted, it may not dilate normally in response to particular visual stimuli. Conversely, in a dimly lit ambient environment, it may be more difficult for the subject's eyes to track the movement of the visual stimuli, or the screening system 100 may be unable to properly detect the subject's ocular response characteristics to the visual stimuli. Thus, in some embodiments, the screening system 100 can measure the amount of environmental light and recommend or effect adjustments for appropriate screening, such as blocking environmental light. In some embodiments, the screening system 100 can additionally be configured to control the amount of light in the test environment, such as by activating or controlling lights in the test environment.

In one embodiment, the screening system 100 could be configured to determine whether the subject has certain conditions, whether preexisting or temporary, that could affect its measurements. For example, the screening system 100 could automatically detect the presence of various indicators (e.g., cataracts), retrieve patient information (e.g., electronic medical records) from a database, or prompt the user to enter such information. This information is important because some indicators (such as cataracts) could affect pupillary response. Pupillary responses may be affected in different ways by different indicators. Thus, in some embodiments, the screening system 100 could be configured to detect and differentiate among such indicators. The screening system 100 could be configured to incorporate the presence of these indicators into the determination of the likelihood that the decrease or lack of ocular response is related to the tracked conditions. In addition, the screening system 100 could be configured to recommend additional screenings in the event that more determinations or more time would be advantageous in achieving a successful screening.

In some embodiments, the screening system 100 could be configured to account for a variety of other events or subject-specific or environmental conditions. For example, the screening system 100 could be configured to detect when the user was startled at the time of the test (e.g., due to a loud sound or bright flash) and adjust environmental conditions or recommend that the subject be retested. In one implementation, the screening system 100 could ask the subject one or more questions, such as “Are you currently suffering from a headache or a lack of sleep?”, prior to beginning the screening test and act accordingly based on the subject's responses (e.g., recommending that the test be postponed). As another example, the screening system 100 could be configured to detect (e.g., via the visual response detector 104) whether there was any motion within the subject's field of vision that could have distracted the subject.

All of the various data associated with the subject that are discussed above, such as the subject's amount of ocular response characteristics at various lighting levels, the subject's response to various tests (e.g., accuracy or timing in performing trail-making tests), and so on, could be stored in a user profile associated with the subject. As discussed above, this data can be stored by and/or retrieved by the screening system 100 (e.g., from the database 110) at the time of the screening test to assist in the determination of positive or negative screening results.

One embodiment for the screening system 100 is shown in FIG. 2. In this embodiment, the screening system 100 could include a headpiece 120 that can be worn on the head 115 of the subject. In the illustrated embodiment, the headpiece 120 includes a holder 121 that is configured to hold a mobile device 122 (e.g., a smartphone or another smart device) that includes a visual response detector 104 (e.g., a camera). The holder 121 can be configured to hold the mobile device 122 such that the visual response detector 104 is oriented towards the subject's face when the headpiece 120 is worn by the subject. The mobile device 122 executing the app can be used to guide the subject through the screening steps with audio, images, text, or combinations thereof. Accordingly, in one implementation, users could place their mobile device 122 in the holder 121, and the mobile device can in turn execute an app stored thereon that performs the screening test, as described herein. In another embodiment, the detector 104 could be integral to the headpiece 120. The headpiece 120 can include one or more straps 124 or other securement devices for securing the headpiece to the subject's head 115 and keep the mobile device 122 in a fixed relationship to the subject's face.

In one embodiment, the headpiece 120 could define a partially or fully enclosed chamber 126 that is configured to provide a fixed environment suitable for the screening test for the subject. The headpiece 120 could further include an air inlet 128 (which can further include a filter 130, such as a P100 filter) and a corresponding outlet 129 for allowing the subject to exhale. The screening test could be activated manually by the subject or automatically by the screening system 100 (e.g., by the software app running on a mobile device 122). In still other embodiments, the headpiece 120 could include earphones 138 to control or reduce environmental noise and direct sounds from the mobile device 122 or sensor to the subject. Such embodiments can be beneficial because they provide a controlled environment for the performance of the screening test, which can increase reliability of the results.

Another embodiment of the screening system 100 is shown in FIG. 3. In this embodiment, rather than using the headpiece assembly described above with respect to FIG. 2, the subject could instead hold their mobile device 122 in close proximity to his or her face (or rest the mobile device 122 in an appropriate location). In this embodiment, the visual response detector 104 could include the onboard camera of the mobile device 122, and the tactile response detector 102 could include the capacitive touchscreen thereof. In this embodiment, the relationship between the subject's head 115 and the mobile device 122 is not fixed, so the mobile device (or the software app executed thereby) can therefore be configured to compensate for motion of the subject relative to the mobile device. Such an embodiment can be beneficial because of its ease of use. In particular, such an embodiment does not require a substantial number of components or for the subject to wear a head assembly or otherwise be within a fixed environment.

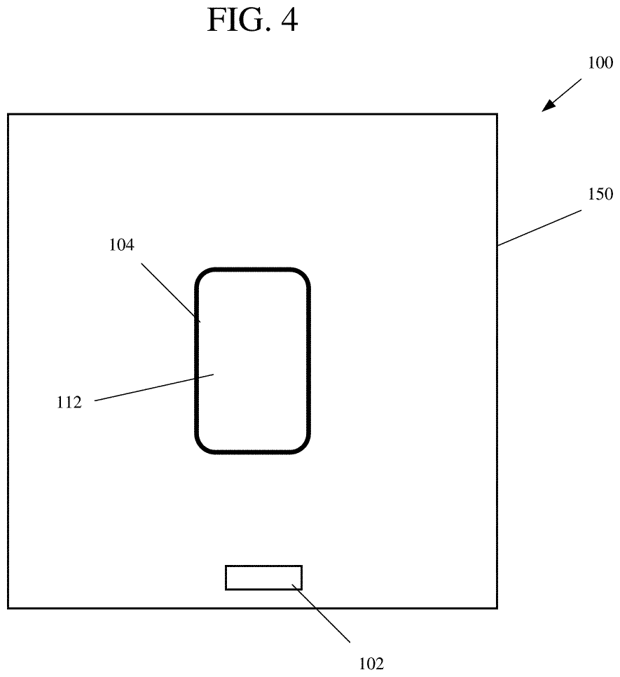

Yet another embodiment of the screening system 100 is shown in FIG. 4. In this embodiment, the screening system 100 is embodied as a high-throughput system that could be suitable for screening at airports, stadia, and so on. In particular, this embodiment of the screening system 100 can include an enclosure 150 into which the subject can enter. The enclosure 150 could be an enclosure that is environmentally controlled, for example. The enclosure 150 could include a complete or partial enclosure (e.g., from the waist up). In such an embodiment, the subject enters the enclosure 150 and faces the visual stimulus source 112, which can further include the tactile response detector 102 (e.g., as a capacitive touchscreen). As noted above, the visual response detector 104 could include a camera or an image sensor, for example. The visual stimulus source 112, visual response detector 104, and/or tactile response detector 102 could be positioned on a wall of the enclosure at a height suitable for visualizing individuals' faces and/or being readily reached by the individuals, for example, or at adjustable heights to optimize the relationship to the user. The visual stimulus source 112 could provide appropriate instructions to the subject (e.g., where to stand), what information to provide to the screening system 100 (e.g., whether the subject has any relevant information that may inform results of screening), or when to exit. The visual stimulus source 112, visual response detector 104, and/or tactile response detector 102 could include a smart device or a specialized sensor apparatus. The visual stimulus source 112, visual response detector 104, and/or tactile response detector 102 could be integral to the enclosure 150 or otherwise located within the enclosure 150.

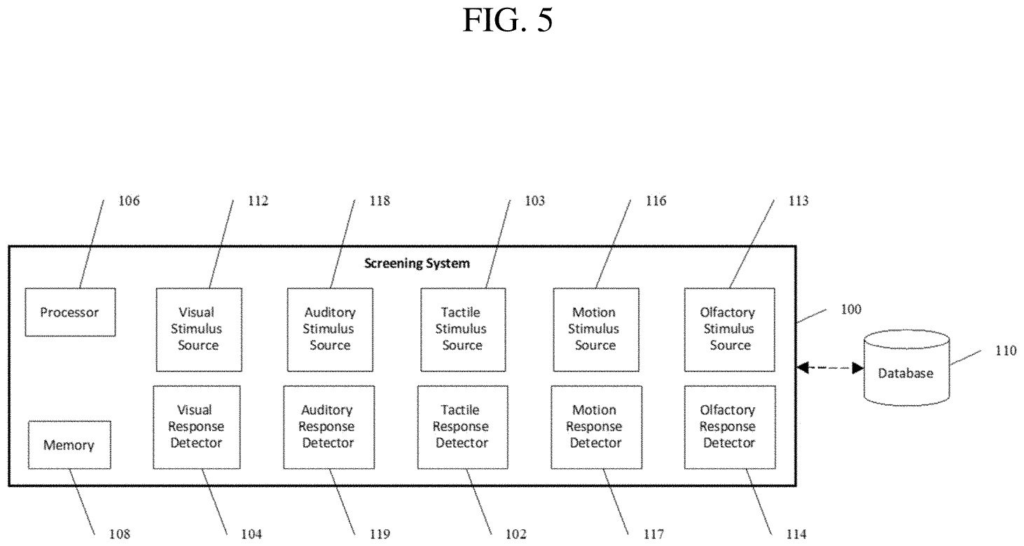

Another embodiment of the screening system 100 is shown in FIG. 5. In this embodiment, the screening system 100 could be embodied as a mobile device (e.g., a smartphone). This embodiment uses emerging capabilities of mobile devices to communicate more effectively with people, especially those with varying abilities, such as hearing, motion and visual challenges, and for additional screening capabilities. In this embodiment, the screening system 100 is configured to provide a variety of different stimuli to the subject and detect the responses by the subject thereto. In particular, the screening system 100 can include a visual stimulus source 112, an auditory stimulus source 118, a visual response detector 104, and a tactile response detector 102, as described above. In this embodiment, the screening system 100 can further include one or more of an auditory response detector 119, a tactile stimulus source 103, an olfactory stimulus source 113, a motion stimulus source 116, a motion response detector 117, and an olfactory response detector 114, including any combination thereof. The auditory response detector 119, such as a microphone, is configured to detect and measure speech and other sounds from the subject, such as verbal responses, slurring of speech, heart beats, and bodily noises. In some embodiment, the auditory response detector 119 can further be configured to detect and measure potentially interfering sounds from the environment. The tactile response detector 102 is configured to detect and measure details of touch, such as, how hard a subject is pressing, how many fingers are touching the detector, details of motion, and uneven touch pressure such as from a tremor. The tactile stimulus source 103 is configured to create the impression of response to touch, such as depressing a virtual key by a subtle motion. The motion stimulus source 116 is configured to generate small motions, such as vibrations, which can be used to assess the ability to detect motion stimuli as well as give feedback to a subject, such as used to get attention when a smart phone is in silent mode. The screening system 100 can further be programmed or otherwise configured to perform speech-to-text translation (e.g., via software or an app executed by the processor 106) to detect and measure the appropriateness and accuracy of verbal responses derived from the speech of the subject. The motion response detector 117, such as an accelerometer, is configured to detect and measure changes in motion exhibited by the subject, such as tremors, startle responses, uneven motions (e.g., due to Parkinson's and other causes), and heartbeats. The method can include other stimuli and detectors, such as electrical stimuli and the associated detectors. The olfactory stimulus source 113 is configured to generate an olfactory stimulus and an olfactory stimulus detector 114 that is configured to detect and measure the same, such as is disclosed in U.S. patent application Ser. No. 17/167,728, titled SYSTEMS AND METHODS FOR SCREENING SUBJECTS BASED ON PUPILLARY RESPONSE TO OLFACTORY STIMULATION, filed Feb. 4, 2021, which is hereby incorporated by reference herein in its entirety.

As noted above, in one embodiment, the screening system 100 can be embodied as a mobile device 122. In other embodiments, the screening system 100 could be embodied as a combination of devices and/or systems that are communicatively coupled. For example, the screening system 100 could include a mobile device 122 that is coupled to a remote computing system (e.g., a server or a cloud computing system). In such embodiments, various steps, aspects, or techniques described above can be collectively executed by or between the devices and/or systems.

### Screening for Neuropathological Conditions

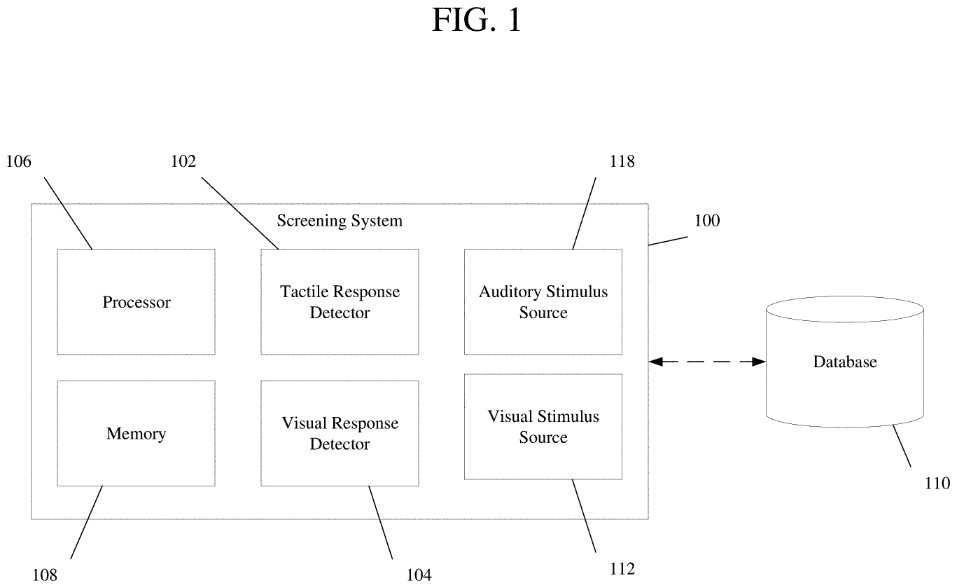

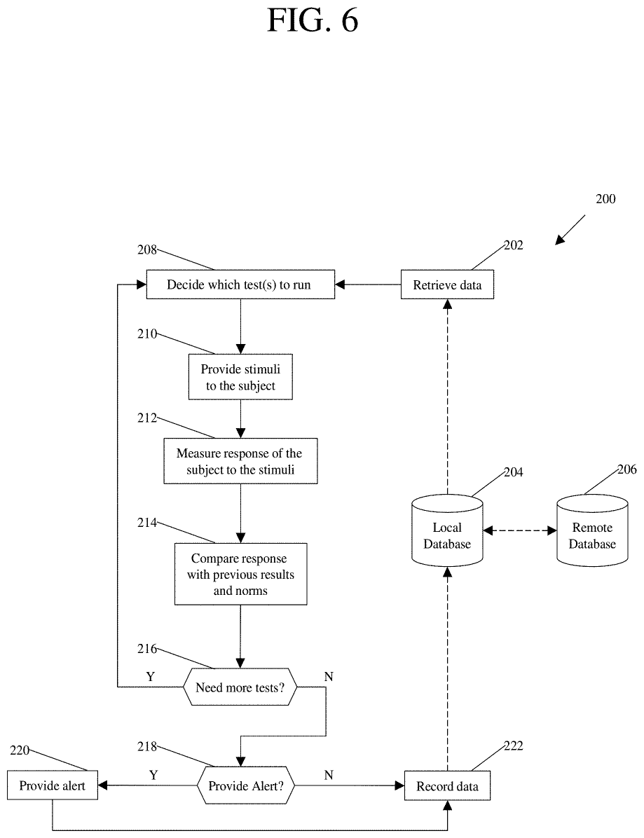

In one embodiment, systems, such as the screening system 100 described above, can be configured to execute various processes for screening subjects for neuropathological conditions based on a variety of different visual-based measures, including the subjects' eye movements characteristics, subjects' eye tracking abilities, subjects' performance on trail-making tests, or subject's eye-hand coordination. One example of such a process 200 is shown in FIG. 6. In the following discussion of the process 200, reference should also be made to FIG. 1. In one embodiment, the process 200 can be embodied as instructions stored in a memory 108 that, when executed by a processor 106, cause the screening system 100 to perform the process. In various embodiments, the process 200 can be embodied as software, hardware, firmware, and various combinations thereof. In various embodiments, the process 200 can be executed by and/or between a variety of different devices or systems. For example, one or more steps of the process 200 could be executed by a mobile device of the screening system 100 and one or more of the remaining steps of the process 200 could be executed by a cloud computing system or another such remote computer system. In various embodiments, the screening system 100 executing the process 200 can utilize distributed processing, parallel processing, cloud processing, and/or edge computing techniques. The process 200 is described below as being executed by the screen system 100; accordingly, it should be understood that the functions can be individually or collectively executed by one or multiple devices or systems.

As generally described above, the screening system 100 can provide various tests to a subject to screen the subject for a variety of different neuropathological conditions. In some embodiments, the screening system 100 can provide a default or predetermined test or set of tests to a subject. In some embodiments, the screening system 100 can select one or more tests to be provided to the subject. For example, the screening system 100 executing the process 200 can determine 208 which test or tests to run, i.e., which test or tests are to be provided to the subject. The screening system 100 can determine 208 which test or tests to provide to the subject based on a variety of different factors, including previous tests provided to the subject or which neuropathological conditions the subject is being screened for. The tests can include, for example, an eye tracking test, a trail-making test, or an eye-hand coordination test.

Accordingly, the screening system 100 can provide 210 one or more visual stimuli to the visual stimulus source 112. In one embodiment, the visual stimuli can be provided via the visual stimulus source 112. In some embodiments, prior to, contemporaneous with, or after measuring 212 the subject's pupil, the screening system 100 can retrieve 202 data associated with the subject, the subject's ambient environment, or other testing parameters (e.g., shutter speed of a camera being used to measure the subject's pupillary response) that could be used to control aspects of the process 200 performed by the screening system 100. In various embodiments, the retrieved 202 data could include a reference against which the subject's measured pupillary response is compared. In one embodiment, the reference could include a default value, such as a preprogrammed value associated with the given neurophysiological stimulus. For example, the screening system 100 could be programmed to store a number of ocular or hand-eye coordination response measurements previously exhibited by the subject and characterize the usual range of ocular responses or hand-eye coordination responses for the subject for the given visual stimulus. In yet another embodiment, the reference could include characterized ocular responses or hand-eye coordination responses of a population of individuals to the visual stimulus. In this embodiment, data from a population of users could be pooled and analyzed to characterize the usual range of ocular or hand-eye coordination responses for particular stimuli. In various embodiments, the retrieved reference or other data could be stored in a profile associated with the subject. In one embodiment, the retrieved data could be stored in a local database 204 (e.g., in the memory 108 of the mobile device 122). In another embodiment, the retrieved data could be stored in a remote database 206 (e.g., a cloud computing system that the mobile device 100 is communicably connectable to).

Accordingly, the screening system 100 can measure 212 the response of the subject to the provided visual stimulus via the visual response detector 104, the tactile response detector 102, or a combination thereof, depending upon the type of test being performed or the type of visual stimulus provided to the subject. Further, the screening system 100 can compare 214 the measured response by the subject to a reference, such as previous results associated with the subject or norms, and determine whether the subject is exhibiting signs of a neuropathological condition. Based on the results of the comparison, the screening system 100 can determine 218 whether to provide an alert to the subject. In one embodiment, the screening system 100 can compare 214 the measured response to a reference for the subject (e.g., which could be included in the retrieved 202 data described above) and determine whether the measured response differs from the retrieved reference. In one embodiment, the screening system 100 may determine whether the measured response differs from the reference by a threshold. For example, in an embodiment where the measured response includes a time delay in a subject's eyes tracking the movement of a visual stimulus, the threshold could be based on the usual time delay exhibited by the subject under prior testing conditions. In another embodiment, the screening system 100 could determine whether the measured response falls outside of a particular range of values associated with the subject's response profile.

If the measured response differs from the reference or the range of reference values, the screening system 100 can provide 220 an alert to the subject. As noted above, if the measured response differs from the response profile, the screening system 100 could recommend medical evaluation and/or testing. In various embodiments, the results of screening could be used to prompt medical evaluation for a variety of different neuropathological conditions (e.g., COVID-19, multiple sclerosis, Alzheimer's disease, or other forms of dementia, for example). The alert could include a push notification provided via a mobile device, a text message, an email, a popup message, and so on. The alert could provide additional recommendations, such as that the user should seek medical evaluation and advice regarding additional testing or whether to take precautionary measures (e.g., self-quarantine or isolation).

If the measured pupillary response does not differ from the pupillary response profile, the screening system 100 could record 222 the data associated with the screening test. In one embodiment, the screening system 100 could likewise record 222 the data associated with the screening test when the measured pupillary response differs from the reference. In one embodiment, the screening system 100 could further add the recorded 222 data to a database (e.g., the local database 204 and/or the remote database 206).

In one embodiment, the screening system 100 executing the process 200 can further determine 216 whether additional testing is required to make a determination regarding whether the subject is exhibiting signs of a neuropathological condition. For example, the screening system 100 could determine 216 that additional testing is required if the screening system 100 determines that testing conditions were compromised (e.g., the screening system 100 detected movement within the subject's field of view via the visual response detector 104). As another example, the screening system 100 could determine 216 that additional testing is required in order to distinguish between different neuropathological conditions. If the screening system 100 determines 216 that additional testing is required, the screening system 100 can automatically begin a new screening test or prompt the subject to manually begin a new screening test. The new screening test performed by a subsequent execution of the process 200 could provide 210 the same or a different visual stimulus to the subject.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. A computer-implemented method for screening a subject for a response to neurophysiological stimuli as an indication for neuropathology associated with COVID-19, the method comprising:
selecting, by a processor of a mobile device, the neurophysiological stimuli from a plurality of different neurophysiological stimuli to provide to the subject based on which of the plurality of different neurophysiological stimuli had been provided to the subject for screening the subject for COVID-19;
presenting, by a display screen of the mobile device, the neurophysiological stimuli to the subject, wherein the neurophysiological stimuli comprise at least one of a trail-making test or an eye-hand coordination test;
measuring, by a detector of the mobile device, a neurophysiological response by the subject to the neurophysiological stimuli;
determining, by the processor of the mobile device, whether a condition affected the measuring of the neurophysiological response;
in response to determining that the condition was present, remeasuring, by the processor, the neurophysiological response by the subject to the neurophysiological stimuli;
determining, by the processor, whether the measured neurophysiological response differs from a reference response by a threshold, whereby such a difference comprises an abnormal response; and
providing, by the processor, an alert for the abnormal response, wherein the alert comprises an intervention associated with COVID-19.

2. The computer-implemented method of claim 1, wherein the measured neurophysiological response of the subject comprises at least one of a change in speed, magnitude, or accuracy.

3. (canceled)

4. The computer-implemented method of claim 1, wherein the neurophysiological response comprises at least one of an eye movement response or a hand motion response.

5. The computer-implemented method of claim 1, wherein the intervention comprises at least one of a recommendation to take a COVID-19 diagnostic test or a recommendation to seek medical evaluation.

6. The computer-implemented method of claim 1, wherein the reference response comprises one or more default values.

7. The computer-implemented method of claim 1, wherein the reference response comprises at least one of a characterized response or a baseline response to the neurophysiological stimuli for the subject.

8. The computer-implemented method of claim 1, wherein the reference response comprises at least one of a characterized response of a population of individuals to the neurophysiological stimuli.

9. A system for screening a subject for a neurophysiological response to neurophysiological stimuli as an indication for neuropathology associated with COVID-19, the system comprising:
a library storing the neurophysiological stimuli; and
a mobile device comprising:
a display screen,
a detector,
a processor, and
a memory coupled to the processor, the memory storing instructions that, when executed by the processor, cause the processor to:
select the neurophysiological stimuli from a plurality of different neurophysiological stimuli to provide to the subject based on which of the plurality of different neurophysiological stimuli had been provided to the subject for screening the subject for COVID-19,
present, via the display screen, the neurophysiological stimuli to the subject, wherein the neurophysiological stimuli comprise at least one of a trail-making test or an eye-hand coordination test,
measure, via the detector, the neurophysiological response by the subject to the neurophysiological stimuli,
determine whether a condition affected the measuring of the neurophysiological response;
in response to a determination that the condition was present, remeasure the neurophysiological response by the subject to the neurophysiological stimuli;
determine whether the measured neurophysiological response differs from a reference response by a threshold, whereby such a difference comprises an abnormal response, and
provide an alert for the abnormal response, wherein the alert comprises an intervention associated with COVID-19.

10. The system of claim 9, wherein the measured neurophysiological response comprises at least one of a change in speed, magnitude, or accuracy of the subject.

11. (canceled)

12. The system of claim 9, wherein the neurophysiological response comprises at least one of an eye movement response or a hand motion response.

13. The system of claim 9, wherein the intervention comprises at least one of a recommendation to take a COVID-19 diagnostic test or a recommendation to seek medical evaluation.

14. The system of claim 11, wherein the alert comprises a push notification.

15. The system of claim 11, wherein the reference response comprises one or more default values.

16. The system of claim 11, wherein the reference response comprises at least one of a characterized response or a baseline response to the neurophysiological stimuli for the subject.

17. The system of claim 11, wherein the reference response comprises at least one of a characterized response of a population of individuals to the neurophysiological stimuli.

18. The computer-implemented method of claim 1, wherein the condition comprises an amount of light in an environment of the subject.

19. The system of claim 9, wherein the condition comprises an amount of light in an environment of the subject.
